SEQUENCE LISTING (2 sequences)

SEQ ID NO: 1 - DNA, 20 bp, Artificial Sequence
  Description of Artificial Sequence Artificially Synthesized Primer Sequence
cttctcctgcaggtgaccat

SEQ ID NO: 2 - DNA, 20 bp, Artificial Sequence
  Description of Artificial Sequence Artificially Synthesized Primer Sequence
agcatgaatcactctgcttc

(19) United States
(12) Patent Application Publication
(10) Pub. No.: US 2004/0265816 A1
(43) Pub. Date: Dec. 30, 2004

(54) Method of judging risk of side effects of remedys for rheumatoid arthritis (ra)

(76) Inventors: Eiichi Tanaka, Tokyo (JP); Naoyuki Kamatani, Tokyo (JP)

Correspondence Address: STERNE, KESSLER, GOLDSTEIN & FOX P.L.L.C., 1100 NEW YORK AVE., N.W., WASHINGTON, DC, 20005, US

(21) Appl. No.: 10/482,689

(22) PCT Filed: Apr. 16, 2002
(86) PCT No.: PCT/JP02/03771

(30) Foreign Application Priority Data
Jul. 5, 2001 (JP) 2001-205100

(51) Int. Cl. C12Q 1/68

(52) U.S. Cl. 435/6

(57) ABSTRACT

The correlation between the diplotype configuration of the NAT2 gene and the adverse effect of SASP in RA patients was assessed. It was discovered that the incidence rate of adverse effects was higher in the patients having no wild type haplotype who were treated with SASP. Thus, the risk of adverse effects of SASP for each individual can be evaluated by determining the diplotype configuration at the NAT2 gene for each subject in terms of the presence of the wild type haplotype.

### TECHNICAL FIELD

The present invention relates to a method for evaluating the risk of adverse effects associated with the administration of sulfasalazine or a pharmaceutical containing same to patients with rheumatoid arthritis.

### BACKGROUND ART

Several prospective randomized clinical trials have shown that sulfasalazine (SASP) is an effective disease-modifying anti-rheumatic drug (DMARD) for the treatment of rheumatoid arthritis (RA) (Hannonen P et al., Arthritis Rheum 36: 1501-1509, 1993; Pinals R S et al. Arthritis Rheum 29: 1427-1434, 1986; Ebringer R et al., J. Rheumatol 19: 1672-1677, 1992). SASP is a pharmaceutical agent widely used as the first or second choice all over the world (McConkey B et al., Br. Med. J. 280: 442-444, 1980; Neumann V C et al., Br. Med. J. 287: 1099-1102, 1983; Pullar T et al., Be. Med. J. 287: 1102-1104, 1983; Farr M et al., Clin. Rheumatol. 3: 473-481, 1984; Bax D E and Amos R S Ann. Rheum. Dis. 44: 194-198, 1985).

Ten to 30% of SASP administered orally is absorbed in the upper gastrointestinal tract and then excreted as is in the urine. The unabsorbed SASP reaches the distal small intestine and colon, and is decomposed there into sulfapyridine (SP) and 5-amino salicylic acid (5-ASA) by the action of bacterial azoreductase (Rains C P et al., Drugs 50: 137-156, 1995). SP is absorbed almost completely in the colon; in the liver, it is not only acetylated but also conjugated with glucuronide and hydroxylated (Rains C P et al., Drugs 50: 137-156, 1995). The acetylation metabolites of SP are mainly excreted in the urine (Rains C P et al., Drugs 50: 137-156, 1995). 5-ASA, by contrast, is excreted directly with the feces or absorbed in the colon. The absorbed 5-ASA is acetylated in the liver, and then excreted mainly in the urine (Rains C P et al., Drugs 50: 137-156, 1995).

The action mechanism of SASP or SP still remains controversial (Rains C P et al., Drugs 50: 137-156, 1995). The proposed mechanisms include immunomodulation, inhibition of folic acid-associated enzyme, anti-bacterial activity, and inhibition of angiogenesis in the synovial membrane. Some studies focused on moieties of SASP that are effective to treat RA (Neumann V C et al., J. Rheumatol. 13: 285-287, 1986; Pullar T et al., Br. Med. J. 290: 1535-1538, 1985; Farr M. et al., Rheumatol Int. 5: 247-251, 1985; Samanta A. et al., Br. J. Rheumatol. 31: 259-263, 1992; Astbury C et al., Br. J. Rheumatol. 29: 465-467, 1990). There are several studies suggesting that SP or acetylated SP (or both) is the moiety responsible for the therapeutic activity (Neumann V C et al., J. Rheumatol. 13: 285-287, 1986; Pullar T et al., Br. Med. J. 290: 1535-1538, 1985; Farr M. et al., Rheumatol Int. 5: 247-251, 1985). However, other studies have proposed that intact SASP is responsible for the therapeutic activity (Samanta A. et al., Br. J. Rheumatol. 31: 259-263, 1992; Astbury C et al., Br. J. Rheumatol. 29: 465-467, 1990). There are also studies suggesting that 5-ASA serves as the active moiety when used to treat inflammatory bowel diseases (Peppercorn M A Ann. Intern. Med. 101: 377-386, 1984; Azad Khan A K et al., 2: 892-895, 1977).

There are various types of possible prognoses after treatment of RA patients with SASP; therefore, each prognosis is difficult to predict. SASP use is limited by its adverse effects. It has been reported that SASP produces adverse effects in about 20 to 30% of RA patients (Amos R S et al., Br. Med. J. 293: 420-423, 1986; Donovan S et al., Br. J. Rheumatol. 29: 201-204, 1990; Felson D T et al., Arthritis Rheum 35: 1117-1125, 1992).

There are several studies on the relationship between SASP efficacy, its adverse effects, and patient acetylator phenotype. In most of the studies, the acetylator phenotype (rapid or slow acetylator phenotype) was determined based on the ratio between acetylated SP and free SP in either serum or urine. Various studies reported that there was no correlation between the concentration of SASP or its metabolites in the serum and either the efficacy or toxicity of SASP (Ropes MW et al., Bull. Rheum Dis. 9: 175-176, 1958; Pullar T et al., Br. J. Rheumatol. 24: 269-276, 1985; Taggart A J et al., Scand J. Rheumatol. Suppl. 64: 29-36, 1987). The correlation between the acetylator phenotype and the efficacy or toxicity of SASP is ruled out in many studies (Pullar T et al., Ann. Rheum. Dis. 44: 831-837, 1985; Kitas G D et al., Scand. J. Rheumatol. 21: 220-225, 1992; Bax D E et al., Br. J. Rheumatol. 25: 282-284, 1986; Chalmers I M et al., J. Rheumatol. 17: 764-770, 1990). However, some reports describe that a specific adverse effect is more often produced in slow acetylators rather than rapid acetylators (Pullar T et al., Ann. Rheum. Dis. 44: 831-837, 1985; Kitas G D et al., Scand. J. Rheumatol. 21:220-225, 1992; Laversuch C J et al., Br. J. Rheumatol. 34: 435-439, 1995).

The acetylation of SASP is catalyzed in the liver by N-acetyltransferase (NAT, BC 2.3.1.5). Blum et al. (1990) cloned three human NAT genes from human leukocyte DNA using a rabbit cDNA encoding arylamine NAT (Blum M. DNA Cell Biol. 9: 193-203, 1990). Two of the three, NAT1 and NAT2, each of which has an 870 bp open reading frame, have been mapped on 8pter-q11. These two have been proven to be functional genes. The third gene was deduced to be a pseudogene.

The NAT2 gene carries some single-nucleotide polymorphisms (SNPs) Deguchi et al. reported for the first time not only the nucleotide sequence of the NAT gene but also the presence of polymorphisms in the gene using human liver samples (Deguchi T et al., J. Biol. Chem. 265: 12757-12760, 1990). Five alleles—namely one wild type allele (WT) and four mutant alleles (M1, M2, M3, and M4)—were reported. Seven different SNP sites were found in the NAT2 gene (Blum M. DNA Cell Biol. 9:193-203, 1990; Deguchi T et al., J. Biol. Chem. 265: 12757-12760, 1990; Bell D A et al. , Carcinogenesis 14: 1689-1692, 1993; Vatsis K P et al., Proc. Natl. Acad. Sci USA 88: 6333-6337, 1991). Five out of the seven resulted in amino acid substitutions, which are: G to A at nucleotide 191 (SNP1: arginine to glutamic acid); T to C at nucleotide 341 (SNP3; isoleucine to threonine); G to A at nucleotide 590 (SNP5: arginine to glutamine), A to G at nucleotide 803 (SNP6: lysine to arginine); and G to A at nucleotide 857 (SNP7: glycine to glutamic acid) (FIG. 1). The two remaining nucleotide substitutions, from C to T at nucleotide 282 (SNP2) and at nucleotide 481 (SNP4), did not result in amino acid substitution (tyrosine and leucine respectively) (FIG. 1). The researchers identified four distinct alleles M1, M2, M3 and M4, in addition to the most typical allele (WT). Each of these alleles contains a specific combination of nucleotide alterations at seven polymorphic sites. Patterns of restriction enzyme digestion for WT, M1, M2, and M3 are shown in FIG. 2. Because each allele is a combination of SNPs at a number of sites, it is considered to be a haplotype. The genetic state of the NAT2 gene of each subject is expressed as a combination of two haplotypes, namely, a diplotype configuration (Bell D A et al., Carcinogenesis 14: 1689-1692, 1993; Blum M et al., 88: 5237-5241, 1991).

The NAT2 activity of a certain subject is associated with the diplotype configuration. Some previous studies have reported that a subject exhibits the rapid acetylator phenotype when it has at least one WT allele, but exhibits the slow acetylator phenotype when it has two mutant alleles (Deguchi T et al., J. Biol. Chem. 265: 12757-12760, 1990; Bell D A et al. , Carcinogenesis 14: 1689-1692, 1993; Blum M et al., 88: 5237-5241, 1991; Hickman D and Sim E Biochem. Pharmacol. 42: 1007-1014, 1991; Cascorbi I et al., Am. J. Hum. Genet. 57: 581-592, 1995). The acetylator type is known to play important roles in the metabolism of agents, such as SASP, isoniazid, phenelzine, hydralazine, procainamide, dapsone, phenelzine, and nitrazepam (Lunde P K et al., Disease and acetylator polymorphism. Adis Health Science Press, 1983; Timbrell J A et al., Clin. Pharm. Therap. 22: 602-609, 1977).

There are some reports regarding the NAT2 acetylator phenotype. Timbrell et al. have reported that hepatotoxicity of isoniazid occurs more frequently in slow acetylators than in rapid acetylators (Harmer D et al., J. Med. Genet. 23: 155-156, 1986). The slow acetylator phenotype for the NAT2 gene is also associated with a higher risk of bladder cancer and a lower risk of colorectal cancer (Roberts-Thomson I C et al., Lancet 347: 1372-1374, 1996). Recently, Inatomi et al. have reported that the haplotype for the NAT2 gene associated with slow acetylation correlates with a high risk of bladder cancer in Japanese (Inatomi H et al., Int. J. Urol. 6:446-454, 1999). The suggestion of a correlation between acetylator phenotype and risk of systemic lupus erythematosus (SLE) following SASP treatment has been controversial (Laversuch C J et al., Br. J. Rheumatol. 34: 435-439, 1995; Baer AN et al., Arthritis Rheum. 29: 508-514, 1986).

As described above, some reports describe a correlation between SASP toxicity and acetylator phenotype in RA patients. However, the relationship between the actual risk of adverse effects in RA patients following SASP administration, and each individual's NAT2 gene diplotype configuration, was unknown. Once the relationship is revealed, it can provide important information necessary to evaluate the appropriateness of SASP administration to RA patients and to select effective therapeutic methods.

### DISCLOSURE OF THE INVENTION

The present invention was achieved through contemplation of the background described above. An objective of the present invention is to elucidate a correlation between the risk of adverse effects associated with administration of sulfasalazine and the diplotype configuration of the NAT2 gene in a patient with rheumatoid arthritis, and to provide a method for evaluating the risk of adverse effects of SASP treatment of individuals based on the correlation.

To achieve the above-described objective, the present inventors studied whether the diplotype configuration of the NAT2 gene correlated with any adverse effects of a therapeutic agent for RA in patients with rheumatoid arthritis (RA). First, the present inventors determined the diplotype configuration of the NAT2 gene (a combination of two haplotypes), based on the genotypic information concerning polymorphic sites in the NAT2 gene, for RA patients. Then, they discovered that adverse effects were experienced by 62.5% of patients having no wild type haplotype who were treated with sulfasalazine (SASP). This incidence was found to be significantly higher (P<0.001) as compared with that in patients having the wild type haplotype (8.1%). Thus, it is reasonable to conclude that an individual having no wild type haplotype is at high risk of adverse effects of SASP. This enables one to predict the risk of adverse effects of SASP administration and to appropriately assess whether SASP may be administered to an RA patient, appropriate doses of SASP, and others.

As described above, the present inventors discovered a relationship between the diplotype configuration of the NAT2 gene and the risk of adverse effects associated with administration of SASP in RA patients. The inventors thus completed the present invention. Specifically, the present invention relates to a method for evaluating the risk of adverse effects of SASP in an individual by determining the diplotype configuration of the NAT2 gene for the subject, and more specifically,

[1] a method for evaluating a subject's risk of developing an adverse effect of a therapeutic agent for rheumatoid arthritis, which comprises the steps of:

(a) determining the diplotype configuration of the N-acetyltransferase 2 (NAT2) gene for the subject; and

(b) judging a subject, who has no wild type haplotype in the diplotype configuration determined in step (a), to be at risk of developing an adverse effect of the therapeutic agent for rheumatoid arthritis;

[2] the method according to [1], wherein the diplotype configuration is determined by a procedure which comprises the steps of:

(a) computing the haplotype frequency of a population based on the genotypic information of the N-acetyltransferase 2 (NAT2) gene for each individual in that population; and

(b) determining the diplotype configuration for a individual based on that individual's genotypic information and the haplotype frequency computed in step (a);

[3] the method according to [2], wherein the haplotype frequency is computed using an EM algorithm;

[4] the method according to [2] or [3], wherein the genotypic information is a polymorphism selected from the group consisting of single-nucleotide polymorphism (SNP), microsatellite polymorphism, and insertion/deletion polymorphism; and

[5] the method according to any one of [1] to [4], wherein the therapeutic agent for rheumatoid arthritis is sulfasalazine.

The present inventors revealed that an individual having no wild type haplotype in the two haplotypes that constitute the diplotype configuration of the N-acetyltransferase 2 (NAT2) gene is at risk for adverse effects of sulfasalazine (SASP), a therapeutic agent for RA. Thus, the present invention provides a method for evaluating the risk of adverse effects of therapeutic agents for RA in an individual, which comprise the step of determining the diplotype configuration at the NAT2 gene for the individual. The adverse effects, the risk of which can be evaluated according to the present invention, include, but are not limited to, for example, anthema, fever, liver function disorders, gastrointestinal disorders, myelosuppression, stomatitis, and edema.

In the present invention, the first step is to determine the diplotype configuration of the N-acetyltransferase 2 (NAT2) gene for each individual (step (a)).

As used herein, the term “haplotype” refers to a combination of alleles at multiple linked sites, such as polymorphic sites. Generally, in any gene region, multiple polymorphic sites, such as single base substitutions (SNP: single nucleotide polymorphism), microsatellites, and insertion/deletion, exist proximal to each other and are linked together to form haplotypes. Every individual carries two haplotypes for each genetic region on the autosomes—one derived from the mother and the other from the father. This combination of two haplotypes of an individual is referred to as the “diplotype configuration”.

The nucleotide sequence of the NAT2 gene, for which the diplotype configuration is to be determined according to the present invention, has been previously reported (See GenBank accession numbers: NM000015 and AF348074; MIM number: MIM243400).

The therapeutic agent for RA, for which the risk of adverse effects can be evaluated by the method of the present invention, includes for example, sulfasalazine and its decomposition product salazine. Sulfasalazine is also called salazosulfasalazine, and is commercially available as an enteric-coated preparation whose trade name is Azulfidine EN and which is generally used to treat RA. Thus, for example, the risk of adverse effects of sulfasalazine when used as an enteric-coated preparation can be evaluated by the method of the present invention. Furthermore, the drug is sometimes used to treat ulcerative colitis. Thus, the adverse effect of the drug when used to treat such diseases can also be evaluated according to the present invention. In addition, salazine, a decomposition product of sulfasalazine, is also used to treat ulcerative colitis, and, thus, the risk of adverse effects of the drug can be evaluated according to the present invention. There is no limitation on the type of administration of each pharmaceutical agent described above in the method of the present invention for evaluating the risk of adverse effects.

The determination of the diplotype configuration of the NAT2 gene for an individual in the above-described step of the present invention can be achieved, for example, by using an individual's genotypic information to calculate haplotype frequency and using this frequency to determine the individual's diplotype configuration; by using a population's haplotype frequency, previously calculated from its genotypic information, to determine the individual's diplotype configuration; or by sequencing after cloning the NAT2 gene to determine an individual's diplotype configuration.

Preferably, the diplotype configuration of the NAT2 gene for an individual can be determined by the method described below. First, the haplotype frequency in the population is computed based on the information on the genotype of the N-acetyltransferase 2 (NAT2) gene for each individual in the population. Then, the diplotype configuration for a subject is determined based on the haplotype frequency computed and the genotypic information for the subject.

The term “genotype” normally refers to a combination of two alleles at a gene locus of an individual. For example, when a polymorphic site exists at a gene locus, a “genotype” indicates the characteristic of the two alleles of an individual corresponding to the polymorphic site. The phrase “genotypic information”, as used herein, refers to the information regarding such genotypes. In the context of the present invention, the genotypic information refers to the information on NAT2 gene. The genotypic information of the present invention may include information on a number of genotypes.

The characteristics of a gene serving as the genotypic information of the present invention include single nucleotide polymorphisms, microsatellites, and mutations such as insertion/deletion. The genotypic information in this invention is not limited to just one type of polymorphism or genetic mutation described above. It may contain several kinds of polymorphisms or genetic mutations. In the method of the present invention, the genotypic information is preferably the genotypic information at the polymorphic sites in the NAT2 gene listed below. However, the information may be the information on the genotypes in loci other than the sites listed. Each numeral indicates a position in the NAT2 gene.

(1) G at 191, (2) C at 282, (3) T at 341, (4) C at 481, (5) G at 590, (6) A at 803, (7) G at 857.

As used herein, the term “population” refers to a group of individuals providing genotypic information. To increase the reliability of diplotype configuration inferred according to the present invention, the population is preferably composed of as many individuals as possible. For example, when the number of subjects in the population is 25 (the number of haplotypes=50) or more and no rare haplotype is contained in the population, a diplotype configuration of an individual can be determined with relatively high accuracy by the present method. Thus, the number of individuals in the population of the present invention is preferably, but not limited to, 25 or greater.

In addition, the population is preferably homogeneous (i.e., comprised of individuals of similar genetic background). For example, when a subject's diplotype configuration is to be determined for a Japanese individual, the “population” used to obtain genotypic information is preferably a Japanese population. The homogeneity of a population can be determined by assessing whether respective gene loci meet the conditions of the Hardy-Weinberg equilibrium. In the present invention, the “population” is preferably a group of RA patients. However, the population from which genotypic information is to be obtained is not limited to such a population because it is empirically understood that haplotype frequency and an individual's diplotype configuration can usually be accurately estimated, even when a population is not uniform or deviates from the Hardy-Weinberg equilibrium. In other words, in the present invention, the “population” is not restricted to any specific population.

When contained in a narrow genetic region, a haplotype rarely changes during alteration of generations because the haplotype changes due to crossover in a genetic region and the probability of crossover within a narrow genetic region is extremely low (i.e., the frequency is once within 100,000 kb in one generation). Therefore, the frequencies of respective haplotypes are often fixed in a particular population, such as the Japanese population. This is referred to as “haplotype frequency”. In most cases, the haplotype frequency varies from population to population.

Any known method can be used to compute the haplotype frequency in the present invention. Examples of preferred methods for computing the haplotype frequency include the maximum-likelihood method based on the EM algorithm (Excoffier, L. & Slatkin, M., 1995; Hawley, M. E. & Kidd, K. K., 1995; Long, J. C., et al., 1995; Fallin, D. & Schork, N. J., 2000); analytical solutions (Elandt-Johnson, R. C., 1971; Hill,_W. G, 1974; Yasuda, N., 1978; Imanishi, T. , et al. , 1991), and sequential haplotype-inferring algorithm (Clark, A. G., 1990).

When the EM algorithm is utilized, a haplotype frequency can be computed from genotypic information, as follows:

Where n denotes the number of loci and aᵢ the number of alleles at the i-th locus, the total number of possible haplotypes at n loci N is represented by N=Πᵢ₌₁ⁿαᵢ.

Suppose g represents the number of individuals without any familial data for whom the data on genotypes at n linked loci can be obtained. A maximum of 2ⁿ⁻¹ types of diplotype configurations may exist for the genotypic data obtained for each individual. Where fᵢ (i=1, 2, . . . , N) denotes the frequency of the i-th haplotype within the population, then fᵢ≧0 (i=1, 2, . . . , N), Σᵢ₌₁ⁿƒᵢ=1.

When the population meets the condition of the Hardy-Weinberg equilibrium, the likelihood for a diplotype configuration of an individual can be computed by giving genotype data of the individual. The procedure of EM algorithm to compute the maximum-likelihood estimate of fᵢ, \hat{ƒ}ᵢ is as follows:

(1) A storage area is configured to store possible haplotypes in a population and suppose the total number of possible haplotypes is T. T=0 at the beginning.

(2) All possible diplotype configurations (combination of two haplotypes; a combination of two identical haplotypes may form one diplotype configuration) are listed for each individual by taking the given genotypes at every locus into consideration.

(3) As a result, the possible haplotype for the individual is determined. If this haplotype does not exist in the storage area, which stores the possible haplotypes in the population, the haplotype is added as a new possible haplotype and the haplotypes in the population are re-numbered. Then, T is appropriately increased.

(4) Next, for this individual, the previously listed possible diplotype configurations are represented by combinations of numbers of the possible haplotypes in the population (a diplotype configuration is a combination of two haplotypes), and the possible diplotype configurations for each individual are stored in the storage area.

(5) Following the listing of the possible diplotype configurations for each individual and possible haplotypes in the population, random positive real numbers are given as variables to fᵢ (i=1, 2, . . . , T). Herein, Σᵢ₌₁^{T}ƒᵢ=1,

where as described above, T denotes the number of all possible haplotypes in the population.

(6) All possible diplotype configurations (given as combinations of two numbered possible haplotypes in the population, as described above) listed. for each individual in (4) consist of two haplotypes. The prior probability for each haplotype in an individual is taken as fi corresponding to the haplotype. Suppose a specific possible diplotype configuration of the individual consists of i-th and j-th haplotypes and that the Hardy-Weinberg equilibrium can be applied, the likelihood for a diplotype configuration of the individual is represented by, when i≠j, 2fᵢfⱼ, and when I=j, fᵢ². The posterior probability for each possible diplotype configuration is computed from the likelihood of all possible diplotype configurations for the individual according to the Bayes' theorem.

(7) The posterior probability of each possible diplotype configuration for each individual is computed from the likelihood of each of the possible diplotype configurations according to Bayes' theorem.

(8) The posterior probabilities of possible diplotype and the joint likelihood are computed for each individual.

(9) The likelihood for all diplotype configurations of each individual are computed by multiplying the joint likelihood of each individual for all the individuals.

(10) The expected values for the numbers of respective haplotypes in the population are computed from the posterior probabilities of possible diplotype configurations for each individual, in consideration of the numbers of the haplotypes forming each diplotype configuration.

(11) Thus computed expected values of the number of each haplotype are respectively divided by 2 g to calculate the expected value for the frequency of each haplotype in the population.

(12) The data of haplotypes giving extremely low frequencies (for example, <10⁻⁴) among the computed haplotype frequencies are removed from the storage area where the possible haplotypes in the population have been stored. Then, the list is rearranged and new number T for the possible haplotypes in the population is configured.

(13) The expected value for the frequency of each haplotype in the population that was computed in (11) is assigned into fᵢ (i=1, 2, . . . , T).

(14) The value determined in (13) is presumed as the frequency of a haplotype in the population, and then the process is resumed from step (6). The steps (6) to (13) are repeated as a cycle. Finally, when all of the likelihoods for the diplotype configurations of each individual calculated in a cycle are extremely low (e.g., only an increase of 10⁻⁵) as compared to that calculated in the previous cycle, the iteration is terminated and the likelihood is assessed to have converged on an approximate value.

(15) The computation described above gives the maximum-likelihood estimates of the frequencies of the haplotypes in the population. Namely, the expected value for the frequency of each haplotype in the population computed in step (11) of the final cycle is the maximum-likelihood estimate.

Not only a haplotype frequency computed by the method described above but also a haplotype frequency previously estimated or determined can be used to determine the diplotype configuration in the present invention.

The “determination of the diplotype configuration” of the present invention includes not only determination of a unique diplotype configuration but also computation of a diplotype distribution that comprises the probabilities of all possible diplotype configurations (posterior probability) for each individual, by statistically predicting the diplotype configuration for each individual based on the genotypic information for each individual.

Any known method can be used to compute the diplotype distribution as described above. Such methods for computing the diplotype distribution include: the Bayes' theorem and calculation of the posterior probability. Specifically, examples of methods for computing the diplotype distribution include those described below. Namely, (1) taking each of the haplotype frequencies computed by the above-described method as a prior probability; (2) performing the computation of steps (6) and (7); and (3) calculating the posterior probabilities for possible diplotype configurations for each individual as the diplotype distribution for each individual.

For example, suppose k-th possible diplotype for an individual as Dₖ, and the combination of haplotypes constituting the diplotype as Hᵢ/Hⱼ. Herein, Hᵢ and Hⱼ denote i-th and j-th haplotypes in a population, respectively. When the maximum-likelihood estimates of the frequencies of the i-th and j-th haplotypes in the population are respectively represented by \hat{ƒ}ᵢ, \hat{ƒ}ⱼ, the likelihood Lₖ for this diplotype configuration Dₖ of this individual can be computed as follows:

$${L}_{k} = \{ \begin{matrix}{\hat{f}}_{i}^{2} & if i = j \\ 2 {\hat{f}}_{i} {\hat{f}}_{j} & if i \neq j\end{matrix} .$$

Suppose the total number of possible diplotype configurations for this individual as M. The posterior probability Pₘ of m-th diplotype configuration Dₘ of the individual is computed by following formula:

Pₘ=Lₘ/Σᵢ₌₁^{M}Lᵢ.

The posterior probability Pₘ is computed for m=1, 2, . . . , M, as the diplotype distribution of this individual.

It has been shown that, in most cases, diplotype distribution generally concentrates in a single diplotype configuration. Thus, when the diplotype distribution computed in the above-described step (b) is concentrated in a single diplotype configuration, the individual is determined to have this concentrated diplotype configuration. Alternatively, when the diplotype distribution is not concentrated in a single diplotype configuration, the probabilities of multiple diplotype configurations for the individual are computed as the diplotype distribution. For example, in an individual, the probability of having diplotype configuration A is computed to be X, and the probability of having diplotype configuration B is computed to be Y.

Alternatively, in the present invention, instead of the haplotype frequencies computed from genotypic information for each individual in a population via the method described above, previously estimated or determined haplotype frequencies can also be used to compute the diplotype distribution.

The “previously estimated or determined haplotype frequency of a population” is preferably the haplotype frequency of a population that includes the individual for whom the diplotype configuration is determined (i.e., computation of diplotype distribution). However, a haplotype frequency computed without the genotypic information for the individual may also be used. According to this method of the present invention, a diplotype configuration of an unknown individual can be determined (i.e., computation of diplotype distribution) based on only the genotypic information of the individual by using previously estimated or determined haplotype frequencies of a population.

The “previously estimated or determined haplotype frequency of a population” may be estimated or determined according to the above-described method of the present invention, for example, those derived from genotypic information of an individual via the maximum-likelihood method based on the EM algorithm.

In the present invention, as the next step, a subject is judged to be at risk for adverse effects of therapeutic agents for RA (step (b)), when the diplotype configuration for the subject determined in step (a) contains no wild type haplotype.

In the present invention, the “wild type haplotype” of NAT2 refers to the nucleotide sequence of the NAT2 gene which comprises: (1) G at 191, (2) C at 282, (3) T at 341, (4) C at 481, (5) G at 590, (6) A at 803, and (7) G at 857. The term “non-wild type haplotype” refers to a sequence which contains a nucleotide residue different from that of the wild type at any of the positions listed above.

If the wild type haplotype is taken as W and a haplotype that is not the wild type is taken as M, there are three types of possible diplotype configurations for an individual, W/W, W/M, and M/M. The term “a haplotype that is not the wild type” refers to a haplotype except for the “wild type haplotype” defined above.

In the present invention, when the diplotype configuration for an individual determined by the method described above is M/M (i.e., having no wild type haplotype), the subject (i.e., individual having no wild type haplotype) is judged to be at risk for adverse effects of a therapeutic agent for RA. If the diplotype configuration for an individual is not concentrated on a single type, then the risk of adverse effects of the therapeutic agent for RA is judged to grow as the posterior probability of M/M increases in the posterior probability distribution of the diplotype configuration for the subject.

The risk of adverse effects in each individual following administration of a therapeutic agent for RA can be predicted using the method of the present invention described above. Thus, based on the information obtained by the method of the present invention, the appropriateness of administration of an RA-treating agent to RA patients, dose of the agent, or such can be assessed properly on a single-patient basis. Thus, by considering an individual's genetic background in this way, an effective treatment can be achieved whereby the adverse effects of SASP are reduced.

### BRIEF DESCRIPTION OF THE DRAWINGS

FIG. 1 shows schematic illustration of the structure of the NAT2 gene.

FIG. 2 is a diagram showing restriction enzyme digestion patterns at three SNP sites for four different types of NAT2 haplotypes. The arrow indicates a restriction site. Each haplotype has a characteristic pattern of restriction enzyme digestion.

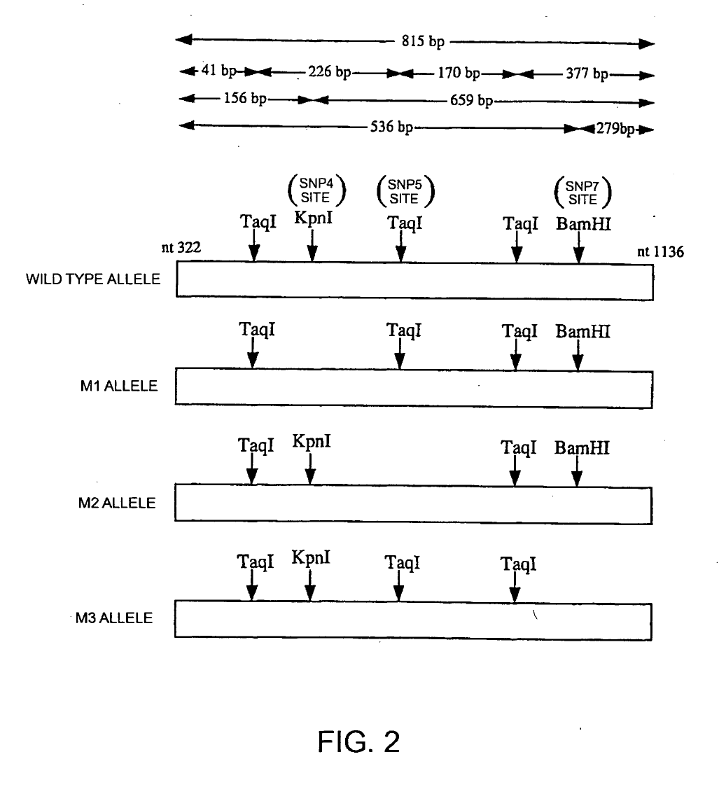

### BEST MODE FOR CARRYING OUT THE INVENTION

The present invention is illustrated in detail below with reference to Examples, but is not to be construed as being limited thereto.

### EXAMPLE 1

Determination of Genotypes and Haplotypes of Patients with Rheumatoid Arthritis (RA)

One hundred and forty-four RA patients (all Japanese nationals: 119 females and 25 males) to whom sulfasalazine (SASP), a therapeutic agent for RA, had been administered, were selected at random from patients in the outpatient department of the Institute of Rheumatology, Tokyo Women's Medical University during 1992 to 1999. All patients had been diagnosed as RA according to the 1987 classification criteria for RA by American College of Rheumatology (formally, American Rheumatism Association) during their clinical courses (Arnett FC et al., Arthritis Rheum. 31: 315-324, 1988).

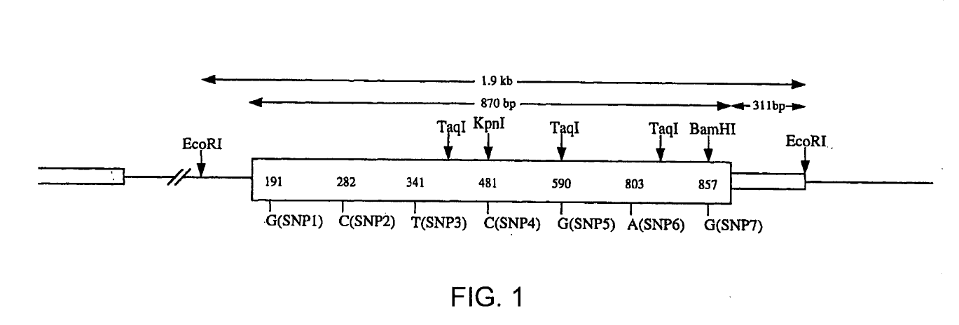

After informed consent, a peripheral blood sample was collected from each patient, and genomic DNA was extracted from mononuclear cells using Wizard Genomic DNA Purification Kit (Promega; cat. # A1620). FIG. 1 shows a schematic illustration of the NAT2 gene. PCR was carried out once using PCR primers (5′-CTT CTC CTG CAG GTG ACC AT-3′/SEQ ID NO: 1 and 5′-AGC ATG AAT CAC TCT GCT TC-3′/SEQ ID NO: 2). The genotype for the three SNP sites at nucleotide 481 (SNP4) 590 (SNP5), and 857 (SNP7) in the NAT2 gene was determined by polymerase chain reaction (PCR)—restriction fragment length polymorphism (RFLP) (FIG. 1). An 815 bp DNA fragment amplified by PCR was digested with any one of KpnI, TaqI, and BamHI. The allele containing C at nucleotide 481 gave 659 bp and 156 bp fragments when digested with KpnI; another allele, containing T at the same site, was resistant to the digestion with the same enzyme. When it had G at nucleotide 590, the 815 bp DNA fragment gave 41 bp, 226 bp, 170 bp, and 377 bp fragments upon digestion; when it had A at the same position, the DNA fragment gave 41 bp, 396 bp, and 377 bp fragments upon digestion. The allele having G at nucleotide 857 contains a BamHI site, and thus gave 536 bp and 279 bp fragments when digested with this enzyme; another allele having A residue at the same position was resistant to the digestion with the same enzyme. Thus, data were obtained, concerning genotypes for the sites at nucleotides 481, 590, and 857 (SNP4, 5, and 7) of NAT2 by the procedure described above.

In addition to the three SNP sites, another four sites (SNP1, 2, 3, and 6) were genotyped for each subject. The genotyping for the four SNP sites of nucleotide 191 (SNP1), 282 (SNP2), 341 (SNP3), and 803 (SNP6) was carried out using a 1211 bp DNA fragment amplified by PCR according to the method of Cascorbi et al. (Cascorbi I et al., Am. J. Hum. Genet. 57: 581-592, 1995).

The alleles, WT, M1, M2, M3, and M4, have many polymorphic sites, and thus are considered as haplotypes. Because each is presumed to be a haplotype, the information on each subject can be described as a diplotype configuration, namely, a combination of two haplotypes. First, the diplotype configuration is determined based on genotypic data concerning SNP4, 6, and 7, and the alleles are referred to as haplotype WT, M1, M2, and M3.

Subsequent analysis was then carried out using the maximum likelihood method described below. If the genotype of a subject comprises each haplotype at every polymorphic site, the diplotype configuration can be determined only when the number of heterozygotic loci is less than two. This is because no phase information is available when only the genotypic information is used. If pedigree information is available, the diplotype configuration can be determined for each subject. When the diplotype information is not available, an alterative estimation method is an expectation maximization algorithm-based maximum likelihood method under the assumption of Hardy-Weinberg equilibrium in the population. This method involves estimating the haplotype frequency in the population from which samples are obtained. Based on the estimated haplotype frequency, the posterior distribution of the diplotype configuration can be computed for each subject. Recent studies have shown that not only the diplotype configuration, but also the estimated haplotype frequency, is correct.

Then, the genotypic data is analyzed using LDSUPPORT program (the method described above) to estimate the haplotype frequency in the population and to compute the posterior probability of diplotype distribution for each patient.

### EXAMPLE 2

Evaluation of the Correlation between the Diplotype Configuration of an RA Patient and the Adverse Effect of SASP Treatment

The treatment with SASP was started at an initial dose of 500 to 1,000 mg/day. Then, the dose of SASP was kept constant or increased up to 1,500 mg/day according to a decision of each physician without strict criterion. Typically, the dose was decided based on the clinical and test data. If the effect of SASP was not enough, the dose was increased. The clinical records were reviewed carefully to assess the adverse effects of pharmaceuticals.

Either Fischer's exact probability test or a chi-square test was used in statistical analysis to assess differences between groups. Odds ratios and 95% confidence intervals (CIs) were determined by computation, if possible.

Previously reported haplotypes and amino acid substitutions are shown in Table 1.

TABLE 1

| | | | | | | | |
|---|---|---|---|---|---|---|---|
| Haplotype | SNP 1 | SNP 2 | SNP 3 | SNP 4 | SNP 5 | SNP 6 | SNP 7 |
| (nt) | (191) | (282) | (341) | (481) | (590) | (803) | (857) |
| WT | G | C | T | C | G | A | G |
| (Amino acid) | (Arg) | (Tyr) | (Ile) | (Leu) | (Arg) | (Lys) | (Gly) |
| M1 | G | C | C | T | G | G | G |
| | | | (Thr) | (Leu) | | (Arg) | |
| M2 | G | T | T | C | A | A | G |
| | | (Tyr) | | | (Gln) | | |
| M3 | G | T | T | C | G | A | A |
| | | | | | | | (Glu) |
| M4 | A | C | T | C | G | A | G |
| | (Glu) | | | | | | |

WT: wild type,
SNP: single-nucleotide polymorphism.

In addition, baseline features of 144 RA patients treated with SASP are shown in Table 2.

TABLE 2

| | total | W/W | W/M | M/M |
|---|---|---|---|---|
| Number of patients | 144 | 73 | 63 | 8 |
| Mean age (years)^{a} | 49.9 ± 13.4 | 48.8 ± 13.7 | 51.3 ± 12.8 | 48.0 ± 15.1 |
| Female (%) | 119 (82.6) | 56 (76.7) | 55 (87.3) | 8 (100) |
| Mean desease duration (months)^{a} | 51.1 ± 65.7 | 33.7 ± 38.4 | 61.6 ± 63.2 | 127.6 ± 165.4 |
| Median disease duration (months)^{a} | 31 | 21 | 50.5 | 62.5 |
| Mean observation period (months) | 27.8 ± 26.8 | 25.1 ± 25.3 | 27.8 ± 27.0 | 7.2 ± 9.9 |
| | (0.03-97) | (0.3-94) | (0.1-97) | (0.03-27) |
| Median observation period (months) | 18 | 16 | 18 | 2.25 |
| Rheumatoid factor positivity (%)^{a} | 125 (86.8) | 64 (87.7) | 53 (84.1) | 8 (100) |
| ESR (mm/hr)^{a} | 51.0 ± 27.0 | 55.0 ± 29.7 | 47.5 ± 23.9 | 37.2 ± 18.7 |
| | (range 3.2-142.0) | (3.2-142.0) | (9.2-114.0) | (3.8-63.8) |
| C-reactive protein (mg/100 ml)^{a} | 2.70 ± 2.98 | 2.82 ± 3.11 | 2.54 ± 2.93 | 2.39 ± 2.27 |
| | (range 0.0-14.0) | (0.0-12.6) | (0.0-14.0) | (0.2-7.7) |
| Corticosteroids users (%)^{a} | 69 (47.9) | 30 (41.1) | 36 (57.1) | 3 (37.5) |
| Number of other DMARDs users (%)^{b} | 39 (27.1) | 20 (27.4) | 17 (27.0) | 2 (25.0) |

*The number of partients to whom other DMARDs had been administered before SASP administrations.
W: wild type haplotype,
M: non-wild type haplotype.
^{a}Data was collected when SASP treatment was started.
^{b}The number of patients to whom other DMARDs had been administered prior to administration of sulfasalazine.

The mean age of patients was 49.9±13.4, and the mean period from the onset of RA to initiation of SASP treatment was 51.1±65.7 months. The period from initiation of SASP treatment to the last occasion of data collection ranged 1 day to 97 months (27.8±26.8 months; median, 18 months). By June 2000, when the last data was collected, the treatment of 83 RA patients (57.6%) with SASP was stopped due to either insufficient or adverse effects. The numbers of RA patients categorized into stage I, II, III, and IV at the start of SASP treatment were 22 (20.7%), 76 (52.9%), 16 (11.3%), and 22 (15.1%), respectively. At that time, 39 patients (27.1%) had been treated with two or more DMARDs. The numbers of patients treated at the initial doses of 500 mg and 1,000 mg were 98 and 46, respectively (Table 3).

TABLE 3

| | Total (%) | W/W (%) | W/M (%) | M/M (%) |
|---|---|---|---|---|
| Initial SASP doses | | | | |
| 500 mg/day | 98 (68.1) | 54 (74.0) | 40 (63.5) | 4 (50.0) |
| 1,000 mg/day | 46 (31.9) | 19 (26.0) | 23 (36.5) | 4 (50.0) |
| Final SASP doses^{a} | | | | |
| 500 mg/day | 39 (27.1) | 19 (26.0) | 18 (28.6) | 2 (25.0) |
| 1,000 mg/day | 100 (69.4) | 50 (68.5) | 44 (69.8) | 6 (75.0) |
| 1,500 mg/day | 5 (3.5) | 4 (5.5) | 1 (1.6) | 0 (0) |

^{a}the doses of SASP when the findings were obtained at the latest occasion or those when SASP administration was discontinued.
W: wild type haplotype,
M: non-wild type haplotype.

During treatment, the highest doses of SASP were 500 mg (39 patients), 1,000 mg (100 patients), and 1,500 mg (five patients). During the treatment with SASP, other DMARDs were used to treat 41 patients (28.5%) because SASP was ineffective.

The test described in this Example is considered as a cohort study rather than case control study because patients with adverse effects and those without adverse effects were not separately selected. In addition, the use of SASP is not influenced by any genetic information (i.e., the physicians are not aware of the genetic information of the patients when they administered SASP to the patients). Thus, in the beginning, both subjects with adverse effects and those without adverse effects were considered as a single group. The possibility that this population is under the condition of Hardy-Weinberg equilibrium is very low. If the adverse effect is influenced by the genotypic information, a group consisting of subjects with adverse effects should not conform to the conditions of the Hardy-Weinberg equilibrium.

In the beginning, 144 patients were analyzed for polymorphisms at three SNP sites (SNP4, 5, and 7). Diplotype configurations were determined by the combined use of the genotypic data at three SNP sites (SNP4, 5, and 7) and reported haplotypes (with a previous established method). Next, the diplotype configuration for each subject was determined by analyzing the same data with LDSUPPORT program (maximum likelihood method). When the maximum likelihood method was used, the probability distribution of diplotype configuration for every individual was concentrated in a single configuration.

Table 4 shows the number and percentage of patients with adverse effects, patients without adverse effects, and total patients.

TABLE 4

| | | | | Without adverse effects | | | With adverse effects | | |
|---|---|---|---|---|---|---|---|---|---|
| Haplotype^{a} | Total (%) | | | of SASP(%) | | | of SASP(%) | | |
| WT GCTCGAG | 208 (72.2) | | | 190^{b} (74.2) | | | 18^{b} (56.3) | | |
| M1 GCCTGGG | 1 (0.3) | | | 1 (0.4) | | | 0 (0) | | |
| M2 GTTCAAG | 55 (19.1) | | | 44^{b} (17.2) | | | 11^{b} (34.4) | | |
| M3 GTTCGAA | 22 (7.6) | {close oversize brace} | 80 (27.8) | 19 (7.4) | {close oversize brace} | 66 (25.8) | 3 (9.4) | {close oversize brace} | 14 (43.8) |
| Mx GTTCGAG | 1 (0.3) | | | 1 (0.4) | | | 0 (0) | | |
| My GCCCAAG | 1 (0.3) | | | 1 (0.4) | | | 0 (0) | | |

^{a}haplotype is represented using seven nucleotide residues at the sites of seven SNPs, namely SNP1, 2, 3, 4, 5, 6, and 7.
^{b}P < 0.05; odds ratio = 2.31; 95% confidence interval = 1.16-4.60.

Among the above haplotypes, four haplotypes had been previously reported. The frequencies of M1, M2, and M3 haplotypes were 0.3%, 19.1%, and 7.6%, respectively. Two haplotypes were combinations of the alleles indicated in Table 4. The haplotypes are referred to as Mx and My, respectively. The total frequency of non-wild type haplotypes was 27.8%. The diplotype distribution for each of 144 subjects was concentrated in a single diplotype configuration. When the wild type haplotype is represented by W and non-wild type haplotype is represented by M, the numbers of subjects having the diplotype configurations W/W, W/M, and M/M were 73, 63, and 8, respectively. The distribution of these diplotype configurations was consistent with the hypothesis of Hardy-Weinberg equilibrium (P>0.05) (Table 4). Table 5 shows the number of subjects having each diplotype configuration.

TABLE 5

| | WT (W) | M1 (M) | M2 (M) | M3 (M) | Mx (M) | My (M) |
|---|---|---|---|---|---|---|
| WT (W) | 72^{a} (50.0%) | 1 (0.7%) | 44 (30.5%) | 18 (12.5%) | 1 (0.7%) | 0 (0%) |
| M1 (M) | | 0 (0%) | 0 (0%) | 0 (0%) | 0 (0%) | 0 (0%) |
| M2 (M) | | | 5 (3.5%) | 0 (0%) | 0 (0%) | 1 (0.7%) |
| M3 (M) | | | | 2 (1.4%) | 0 (0%) | 0 (0%) |
| Mx (M) | | | | | 0 (0%) | 0 (0%) |
| My (M) | | | | | | 0 (0%) |

The table shows the number and percentage of subjects having each diplotype configuration determined from the NAT2 haplotypes.
WT, W: wild type; M: non-wild type haplotype.
^{a}73 subjects have the WT/WT (=W/W) diplotype configuration.

The aim of this cohort study is to detect differences in the frequency of adverse effects of SASP among RA patients having different diplotype configurations. Therefore, it is important to exclude the possibility that the baseline features are quite different among patients having different diplotype configurations. Table 1 indicates that there is no significant difference in the baseline features among patients having different diplotype configurations. Table 2 indicates that there is no significant difference in the initial and final doses of SASP used among patients having different diplotype configurations.

Sixteen patients (11.1%) experienced the adverse effects of SASP. Table 6 shows the numbers of patients who experienced the respective adverse effects. It should be noted that some patients experienced a number of adverse effects.

TABLE 6

| | | Number of discontinued |
|---|---|---|
| Adverse effects | n^{a} = 144 (%) | administration^{b} |
| Rash | 12 (8.3) | 11 |
| Fever | 9 (6.3) | 9 |
| Increase in the level | 5 (3.5) | 4 |
| of transaminase | | |
| Gastrointestinal symptoms | 3 (2.1) | 3 |
| Myelosuppression | 3 (2.1) | 3 |
| Stomatitis | 2 (1.4) | 1 |
| Edema | 1 (0.7) | 1 |

Sixteen patients (11.1%) experienced the adverse effects of SASP. One patient experienced two or more types of adverse effects of SASP.
^{a}the number of RA patients treated with SASP.
^{b}the number of RA patients in whom SASP treatment was discontinued because of the adverse effects.

Table 7 shows detailed clinical data as well as diplotype configurations for the 16 patients who experienced the adverse effects.

TABLE 7

| | Age(years)/ | Duration1^{a} | | Initial | Duration2^{c} | Adverse | Withdrawal | Hospital- | Diplotype |
|---|---|---|---|---|---|---|---|---|---|
| Patient | Gender | (months) | Complication | dose^{b} | (weeks) | effects | of SASP | ization | configuration |
| 1 | 59/F | 8 | — | 500 | 2 | R, S | + | − | WT/WT (W/W) |
| 2 | 46/F | 130 | — | 500 | 1 | F | + | − | WT/WT (W/W) |
| 3 | 44/F | 3 | — | 1,000 | 2 | R | + | − | WT/WT (W/W) |
| 4 | 16/F | 6 | SLE, Sjs | 500 | 2 | F, R, T, M | + | + | WT/WT (W/W) |
| 5 | 50/F | 2 | Sjs | 500 | 8 | R | + | − | WT/WT (W/W) |
| 6 | 26/F | 40 | — | 1,000 | 2 | F, R | + | − | WT/WT (W/W) |
| 7 | 55/F | 1 | — | 1,000 | 4 | R | − | − | WT/WT (W/W) |
| 8 | 59/F | 4 | — | 500 | 2 | F, R | + | + | WT/M2 (W/M) |
| 9 | 61/F | 78 | — | 500 | 120 | S | − | − | WT/M2 (W/M) |
| 10 | 49/F | 1 | — | 500 | 2 | F, R, E | + | − | WT/M2 (W/M) |
| 11 | 65/F | 87 | — | 1,000 | 1 | GI | + | − | WT/M3 (W/M) |
| 12 | 58/F | 57 | Hyperthyroidism | 500 | 1 | F, R, T, | + | + | M2/M2 (M/M) |
| | | | | | | GI, M | | | |
| 13 | 31/F | 0 | Sjs, Hypothyroidism | 1,000 | 2 | F, R, T | + | + | M2/M2 (M/M) |
| 14 | 35/F | 113 | — | 500 | 1 | F, R, GI | + | − | M2/M2 (M/M) |
| 15 | 69/F | 242 | — | 500 | 4 | T | − | − | M2/M2 (M/M) |
| 16 | 59/F | 52 | — | 1,000 | 2 | F, R, T, M | + | − | M3/M3 (M/M) |

SLE, systemic lupus erythematosus; Sjs, Sjögren's syndrome; R, rash; F, fever; T, increase in the level of transaminase; GI, gastrointestinal symptoms; S, stomatitis; M, myelosuppression; E, edema; WT, wild type; W, wild type haplotype; M, non-wild type haplotype.
^{a}Duration 1 = the period between RA onset and the initiation of SASP treatment.
^{b}Initial dose = the initial dose of SASP.
^{c}Duration 2 = the period between sulfasalazine treatment and experience of the adverse effects.

Most adverse effects were experienced within two weeks of the start of SASP treatment (Table 7). The proportion of each NAT2 gene haplotype was compared between patients with adverse effects and those without adverse effect. Differences were found in the proportions of WT and M2 between patients with adverse effects and those without adverse effect Specifically, the proportion of M2 was significantly higher in the group of patients with adverse effects (Table 4). Table 8 shows the numbers of patients whose diplotype configurations were W/W, W/M, and M/M, and the numbers of patients with adverse effects and those without adverse effect among 144 patients.

TABLE 8

| | | | | Number of subjects | | | Number of subjects | | |
|---|---|---|---|---|---|---|---|---|---|
| Diplotype | | | | without adverse | | | with adverse | | |
| configuration | Total | | | effects (%) | | | effects (%) | | |
| W/W | 73 | | | 66 | | | 7 | | |
| | | {close oversize brace} | 136 | | {close oversize brace} | 125^{a} (91.9) | | {close oversize brace} | 11^{a} (8.1) |
| W/M | 63 | | | 59 | | | 4 | | |
| M/M | 8 | | | 3^{a} (37.5) | | | 5^{a} (62.5) | | |

| | | | |
|---|---|---|---|
| Total | 144 | 128 (88.9) | 16 (11.1) |

^{a}P < 0.001; odds ratio = 7.73; 95% confidence interval = 3.54-16.86.

It should be noted that all non-wild type haplotypes are represented by M whereas W denotes the wild type haplotype. For all patients, the number of subjects having the diplotype configurations W/W, W/M, and M/M were 73, 63, and 8, respectively. The data did not indicate a departure from the Hardy-Weinberg equilibrium (p>0.05, chi-square test). Then, the proportions of diplotype configurations were compared between the two groups. Table 7 shows that adverse effects were produced at higher incidence rates in the group of patients having the diplotype configuration M/M than those having W/W (p<0.01; Fisher's exact probability test) and W/M (p<0.001; Fisher's exact probability test). There was no difference in the incidence rates of adverse effects between the groups of patients having the diplotype configurations W/W and W/M (p=0.357; Fisher's exact probability test). The patients were classified into two groups of patients having the wild type haplotype and patients having no wild type haplotype. The rate of adverse effects was 62.5% in the former group, and 8.1% in the latter group (p<0.001; Fisher's exact probability test; RR=7.73; 95% CI=3.54-16.86) (Table 8).

Due to the severity of adverse effects, four patients were hospitalized and treated with corticosteroid (Table 7). The proportion of patients with severe adverse effects was very small (1.5%) in the group of patients having the wild type haplotype (W/W or W/M), but was considerably higher (25.0%) in the group of patients having no wild type haplotype (M/M) (Table 9). The difference was statistically significant (p<0.05; Fisher's exact probability test; RR=17.0; 95% CI=2.74-105.5). An explicit difference was found in the incidence rate of adverse effects between the groups of subjects having different diplotype configurations.

TABLE 9

| Diplotype | Number of subjects without | | | Number of subjects with | | |
|---|---|---|---|---|---|---|
| configuration | severe adverse effects (%) | | | severe adverse effects (%) | | |
| W/W | 72 | | | 1 | | |
| | | {close oversize brace} | 134^{a} (98.5) | | {close oversize brace} | 2^{a} (1.5) |
| W/M | 62 | | | 1 | | |
| M/M | 6^{a} (75.0) | | | 2^{a} (25.0) | | |

| | | |
|---|---|---|
| Total | 140 (97.2) | 4 (2.8) |

^{a}P < 0.005; odds ratio = 17.0; 95% confidence interval = 2.74-105.5.

Industrial Applicability

The present invention provides a method for evaluating an individual's risk of adverse effects of sulfasalazine (SASP), which is a therapeutic agent for RA. The evaluation method of the present invention allows each individual's risk of adverse effects to be predicted prior to SASP administration. Thus, before SASP administration, physicians can properly assess the appropriateness of SASP administration, doses, or such by determining the diplotype configuration for each RA patient through genotyping the NAT2 gene. Thus, the risk of adverse effects can be reduced, and RA patients can be treated effectively.

1. A method for evaluating a subject's risk of developing an adverse effect of a therapeutic agent for rheumatoid arthritis, which comprises the steps of:
determining the diplotype configuration of the N-acetyltransferase 2 (NAT2) gene for the subject; and
judging a subject, who has no wild type haplotype in the diplotype configuration determined in step (a), to be at risk of developing an adverse effect of the therapeutic agent for rheumatoid arthritis.

2. The method according to claim 1, wherein the diplotype configuration is determined by a procedure which comprises the steps of:
computing the haplotype frequency of a population based on the genotypic information of the N-acetyltransferase 2 (NAT2) gene for each individual in that population; and
determining the diplotype configuration for an individual based on that individual's genotypic information and the haplotype frequency computed in step (a).

3. The method according to claim 2, wherein the haplotype frequency is computed using an EM algorithm.

4. The method according to claim 2 or 3, wherein the genotypic information is a polymorphism selected from the group consisting of single-nucleotide polymorphism (SNP), microsatellite polymorphism, and insertion/deletion polymorphism.

5. The method according to claim 1, wherein the therapeutic agent for rheumatoid arthritis is sulfasalazine.

6. The method according to claim 2, wherein the therapeutic agent for rheumatoid arthritis is sulfasalazine.

7. The method according to claim 3, wherein the therapeutic agent for rheumatoid arthritis is sulfasalazine.

8. The method according to claim 4, wherein the therapeutic agent for rheumatoid arthritis is sulfasalazine.
